(19) United States
(12) Patent Application Publication
(10) Pub. No.: US 2004/0006225 A1
(43) Pub. Date: Jan. 8, 2004

(54) Preparation of enantiomerically enriched amine-functionalized compounds

(76) Inventors: Marcelles Sluis Van Der, Groningen (NL); Quirinus Bernardus Broxterman, Munstergeleen (NL); Ben Lange De, Munstergeleen (NL)

Correspondence Address: Kate H Murashige, Morrison & Foerster, Suite 500, 3811 Valley Centre Drive, San Diego, CA, 92130-2332, US

(21) Appl. No.: 10/297,023

(22) PCT Filed: May 21, 2001
(86) PCT No.: PCT/NL01/00384

(30) Foreign Application Priority Data
May 26, 2000 (NL) 1015314

(51) Int. Cl. C07D 265/30; C07D 277/04; C07D 263/04; C07D 211/56; C07D 207/34

(52) U.S. Cl. 544/59; 544/162; 544/382; 546/244; 548/190; 548/233; 548/321.5; 548/557; 562/401; 564/336

(57) ABSTRACT

Process for removing a residual fragment of a chiral auxiliary from a diastereomeric compound with formula (2)

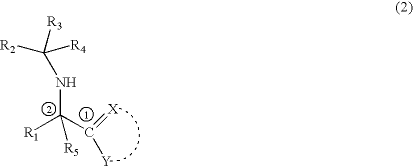

in the preparation of an enantiomerically enriched, amine-functionalized compound, with the diastereomeric compound being subjected to a non-reductive removal of the residual fragment of the chiral auxiliary, the carbon atom that is removed having an oxidation state of +3 which is not lowered during the method of removal.

The chiral auxiliary is preferably chosen from the group of amides or esters of proteogenous amino acids or from the group of phenylglycine amide, an ester of phenylglycine, p-OH-phenylglycine amide, an ester of p-OH-phenylglycine, α-methylphenylglycine amide and an ester of α-methylphenylglycine.

The invention relates to a process for removing a residual fragment of a chiral auxiliary in the preparation of an enantiomerically enriched, amine-functionalized compound with formula 1

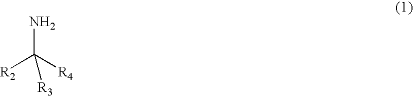

in which R₂, R₃, R₄ are each different and stand for H, a substituted or unsubstituted (cyclo)alkyl group, alkenyl group, aryl group, cyclic or non-cyclic heteroalkyl group or heteroaryl group with one or more N—, O—of S-atoms, or (CH₂)ₙ—COR₆, where n=1, 2, 3 . . . 6 and R₆=OH, a substituted or unsubstituted alkyl group, aryl group or alkoxy group, with for example 1-20 C-atoms or a substituted or unsubstituted amino group, in which a diastereomeric compound with formula 2

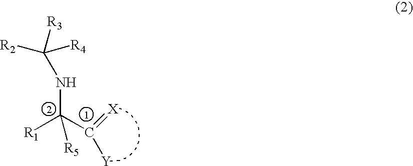

in which R₂, R₃ and R₄ are as defined above, and R₁ and R₅ are each different and R₁ stands for a modified or unmodified side chain of a proteogenous amino acid or a substituted or unsubstituted phenyl group, R₅ stands for H or a lower alkyl group, for example a C₁-C₅ alkyl group, and in which X=O and Y=OR, where R represents H or a C₁-C₇ alkyl group, or Y=NR₇R₈, where R₇ and R₈ each independently represent H, a (cyclo)alkyl group, alkenyl group or aryl group, with for example 1-20 C-atoms, or X and Y together stand for N, is subjected to a non-reductive removal of the residual fragment of the chiral auxiliary, the carbon atom that is removed having an oxidation state of +3 which is not lowered during the method of removal.

In the literature examples are known of processes in which an enantiomerically enriched chiral auxiliary, for example an enantiomerically enriched valine ester, is applied in the preparation of enantiomerically enriched compounds; see for example Basile, T et al; J. Org. Chem., 59 (25), 7766-7773 (1994). As starting material use is made of a suitable prochiral compound, corresponding to the desired enantiomerically enriched compound, and an enantiomerically enriched ester of valine as chiral auxiliary. In the known process a diastereomeric compound is formed which, after removal of the residual fragment of the chiral auxiliary, yields the desired enantiomerically enriched compound.

In these known processes the removal of the residual fragment of the chiral auxiliary usually takes place via reduction of the ester with hydrides, for example LiAlH₄ or NaBH₄, to the corresponding amino alcohol, followed by oxidative splitting with for example H₅IO₆/CH₃NH₂, as described in the above-mentioned reference. However, the complex hydrides used are rather expensive and require extra safety precautions when used on a large scale. Moreover the costs of the oxidative splitting are rather high.

The invention now provides a route to enantiomerically enriched compounds that does not give rise to the above-mentioned disadvantages.

Applicant has now found a very attractive route, based on the use of amino acids or derivatives thereof as chiral auxiliary, in which the residual fragment can be removed in a simple manner without using the known reducing and/or oxidizing agents. Preferably use is made of derivatives, in particular amides or esters, of inexpensive amino acids, in particular proteogenous amino acids, for example aspartic acid, glutamic acid, methionine or valine, or inexpensive, non-proteogenous amino acids, for example phenylglycine, p-hydroxyphenylglycine or α-methylphenylglycine, as a chiral auxiliary.

The preparation of enantiomerically enriched compounds with formula 2 can be carried out according to known chemical conversions. Compounds with formula 2 can be prepared by converting an enantiomerically enriched amino acid derivative with formula 4

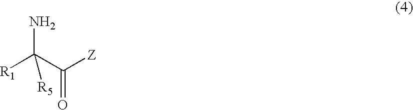

in which R₁ and R₅ have the above-mentioned meanings and in which Z stands for OH, a C₁-C₇ alkoxy group or NR₇R₈, where R₇ and R₈ each independently represent H, a (cyclo)alkyl group, alkenyl group or aryl group, with for example 1-20 C-atoms, with the aid of a compound with formula 5

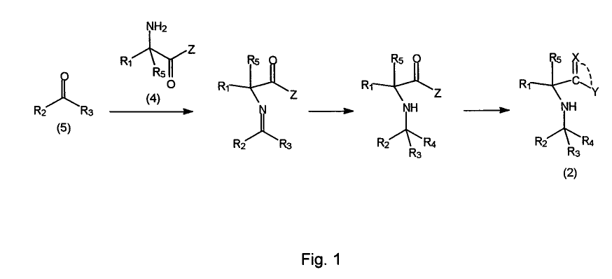

R₂—C(O)—R₃ (5)

where R₂ and R₃ have the above-mentioned meanings, into the corresponding Schiff base (imine) and subsequently converting the resulting Schiff base into the enantiomerically enriched compound with formula 2 with the aid of a reducing agent or an organometallic compound (see FIG. 1). When use is made of an amino acid as chiral auxiliary, the Schiff base will usually be prepared and used as the carboxylic acid salt.

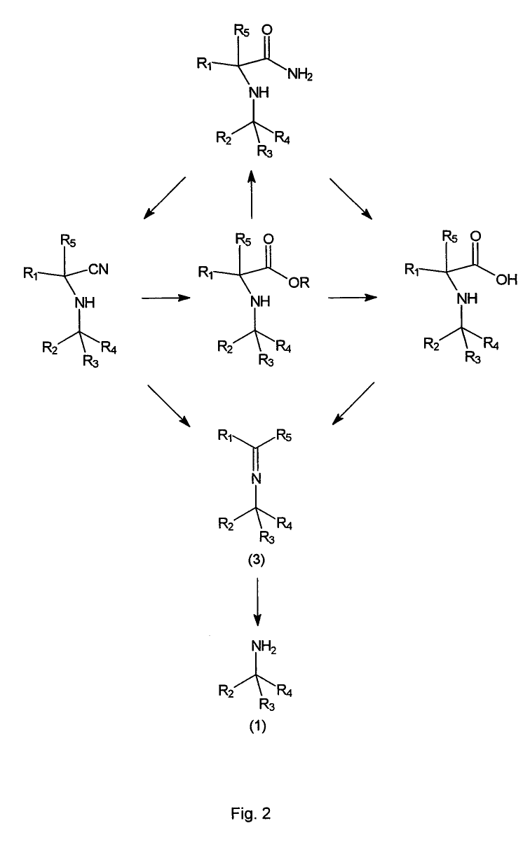

In the process according to the invention the residual fragment is removed from the chiral auxiliary without the carbon atom of the amino acid fragment that is removed—indicated by \mathring{1} in formula 2—being reduced. In the course of the removal of the residual fragment a compound with formula (3) can be formed

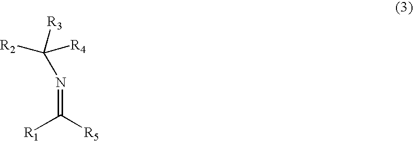

in which R₁, R₂, R₃, R₄ and R₅ are as described above. This compound with formula (3) can subsequently be converted in a known way into the corresponding amine-functionalized compound with formula 1; see for example FIG. 2.

When an amino acid amide of which the amide group is not substituted (with R₇ and R₈ equal to H) is used as chiral auxiliary, the residual fragment of the chiral auxiliary can for example be removed via dehydration of the amide group to a nitrile according to known methods, for example as described in J. March, Advanced Organic Chemistry, 4^{th} Ed. Wiley-lnterscience, New York 1992, 1041-1042. The dehydration may be performed by treating the amide with SOCl₂, POCl₃, PCl₅, p-TosCl/pyridine, Tf₂O/pyridine or with the Vilsmeier reagent in combination with an organic or inorganic base. The Vilsmeier reagent can be prepared by reacting dimethylformamide (DMF) with oxalylchloride in acetonitrile, dichloromethane, chloroform, dioxane, tetrahydrofuran (THF), or diethylether. In a general procedure, the Vilsmeier reagent is formed in the desired solvent for instance at a temperature between 0° C. and room temperature. The formation normally will be completed in 5-15 minutes. In a preferred embodiment a solution of the amide in the desired solvent is added dropwise to the Vilsmeier reagent at a temperature between 0° C. and room temperature. The addition normally will be completed in 10-20 minutes. For the formation of the nitrile, two equivalents of a base are added. Preferably an organic base, for instance pyridine or triethylamine (TEA) is used. Inorganic bases may also be effective. The deprotection procedure proceeds in most cases with retention of configuration at the newly created stereocenter. More in particular, applicant has found that dehydration of the amide with the aid of oxalyl chloride/DMF with pyridine as base at room temperature took place almost quantitatively. The aminonitrile obtained can subsequently be converted into the corresponding imine via a retro-Strecker reaction, for example by treatment at a high temperature, for example between room temperature and reflux temperature of the chosen solvent. Suitable solvents that can be used are, any inert solvents in which reasonable amounts of all reaction components resolve. Treating the nitrile with an organic or inorganic base in a protic solvent also results in elimination of HCN. In a preferred embodiment, the nitrile is added to a suspension of 1.5-3 equivalents of, for instance, KOH or K₂CO₃ in ethanol. Refluxing the mixture for about 1-3 hours results in full elimination of HCN. Examples of suitable bases are (earth)alkali metal hydroxides, (earth)alkalimetal carbonates, and organic bases for instance tertiary amines. Alternatively, short heating at high temperature (>100° C.) and reduced pressure of the crude imine is also possible. The optimum temperature and pressure depends on the reaction system involved and can be easily determined by the skilled person. As an example, usually the conversion of the nitrile to the imine at a temperature of 160° C. will take several minutes.

The imine can subsequently be converted into the corresponding amine-functionalized compound according to known methods, for example by treatment with an aqueous strong acid at elevated temperature, for example between room temperature and reflux temperature, for example with 30% HCl. Another general method is for example the transfer of the imine-carbon containing fragment to a hydrazine or an oxime. It was found that for example treatment with hydroxylamine in a water/tetrahydrofuran (THF) mixture or with phenylhydrazine in hexane was a particularly mild method that yielded the amine-functionalized compound almost quantitatively.

When use is made of an amino acid amide as chiral auxiliary it also appeared possible to first hydrolyze the amide group according to known methods for amide hydrolysis, such as acid, alkaline, enzymatic or oxidative hydrolysis, to form the corresponding carboxyl group, for example by treatment with an aqueous strong acid, for example 15%-30% HCl at elevated temperature, for example between room temperature and reflux temperature, followed by a reaction that, overall, leads to removal of the CO₂ group. In one embodiment a solution of the amide in aqueous HCl is refluxed overnight. Neutralization of the cooled solution with a base, for instance aqueous NaOH, results in precipitation of the amino acid. Alternatively, the amide is treated with Na₂O₂ in water, as for instance described in Vaughn, Robbins, J. Org. Chem., 1975, 40, 1187. Refluxing the reaction mixture overnight, quantitatively gives the amino acid. The latter method gives better results due to less decomposition. The CO₂ removal for example can take place via decarbonylation or decarboxylation to form the imine, for example by conversion of the carboxyl group into a group that is easily removed, for example mesylate, tosylate or acid chloride, followed by treatment with a base. A specific example is the conversion with the aid of pyridine/p-toluene-sulphonyl chloride, as described in J. C. Sheehan, J. W. Frankenfeld, J. Org. Chem. 1962, 27, 628-629. It was found that a particularly mild embodiment of the removal was the treatment with oxalyl chloride/dimethylformamide (DMF) (Vilsmeier reagent) with triethylamine as base, or with trifluoromethane sulphonic acid anhydride and triethylamine. Alternative methods are for example the treatment with SOCl₂, PCl₅, p-TosCl of COCl₂ and triethylamine. This reaction can be performed e.g. in non-protic solvents for instance acetonitrile, dichloromethane, chloroform, dioxane, THF, or diethylether. Of course it is also possible to choose a non-nucleophilic base other than triethylamine. The imine can subsequently be converted as described above into the corresponding amine-functionalized compound.

When an amino acid ester is used as chiral auxiliary, the residual fragment can for example be removed via conversion with ammonia to form the corresponding amino acid amide, which subsequently can be converted into the corresponding amine-functionalized compound in one of the ways described above.

When an amino acid ester is used as chiral auxiliary, the chiral amine-functionalized compound can also be obtained by hydrolysis of the ester to the acid, for example by treatment with acid, followed by removal of the residual fragment and conversion of the imine into the corresponding chiral amine, as described above. The ester may, for instance, be converted into the amino acid under standard hydrolysis condition, for instance by stirring the ester in a methanolic solution of NaOH, for instance at room temperature for 3 days. The deprotection procedure proceeds in most cases with retention of configuration at the newly created stereocenter.

When an amino acid amide of which the amide group is not substituted (R₇═R₈═H) is used as chiral auxiliary, the residual fragment of the chiral auxiliary can be converted into an aminonitrile via dehydration, as described above, and subsequently, via treatment with an alcohol and an acid (for example with methanol/HCl), be converted into the amino acid ester, which subsequently can be converted into the corresponding chiral amine-functionalized compound as described above.

The invention will now be elucidated on the basis of examples without however being limited by these.

### EXAMPLES

### Example I

Synthesis of the imine of (R)-phenylglycine amide and isobutyraldehyde

3.6 g (50 mmol) of isobutyraldehyde and 0.7 g 4 Å sieves were added to 7.5 g (50 mmol) of (R)-phenylglycine amide in 50 ml of dichloromethane. The mixture was stirred for 4 hours at 20-25° C. After filtration the solution was evaporated.

10.6 g (45.0 mmol, 95%) of the Schiff base of (R)-phenylglycine amide and isobutyraldehyde was obtained as a white solid.

¹H NMR (200 Mhz, CDCl₃): 1.06 (m, 6H), 2.46 (m, 1H), 4.67 (s, 1H), 5.68 1H), 6.90 (s, 1H), 7.21-7.37 (m, 5H), 7.60 (d, J=4.4, 1H).

¹³C NMR (50 MHz, CDCl₃): 173.4, 170.9, 137.9, 127.1, 126.2, 125.6, 75.2, 32.7, 17.4, 17.3.

### Example II

Allylation of the imine of (R)-phenylglycine amide and isobutyraldehyde

2.4 g (20 mmol) of allyl bromide was added with stirring to a mixture of 4.1 g (20.0 mmol) of the Schiff base of (R)-phenylglycine amide and isobutyraldehyde and activated Zn (2 eq) in 100 ml dry THF, upon which an exothermic reaction took place. The mixture was stirred for 1 hour at 20-25° C. and subsequently 100 ml of a saturated solution of NaHCO₃ in water was added. 100 ml of ethyl acetate was added to this. After separation of the ethyl acetate layer, the aqueous layer was again extracted with 100 ml ethyl acetate. After drying on MgSO₄, filtration and evaporation, 3.8 g (15.4 mmol, 77%) of the homoallylamine was obtained as a yellow oil.

¹H NMR (200 MHz, CDCl₃): 0.72 (d, J=6.9 Hz, 3H), 0.85 (d, J=6.6 Hz, 3H), 1.87 (m, 2H), 2.17 (m, 1H), 2.37 (m, 1H), 4.25 (s, 1H), 5.03 (s, 1H), 5.07 (d, J=10.6 Hz, 1H), 5.76 (m, 1H), 6.02 (bs, 1), 7.20-7.34 (m, 6H).

¹H NMR yielded only one stereoisomer.

¹³C NMR (50 MHz, CDCl₃): 174.0, 137.2, 134.0, 126.3, 125.5, 125.0, 114.8, 62.4, 58.5, 32.2, 26.8, 16.5, 14.5.

### Example III

Dehydration of homoallylamine from Example II to Form the nitrile, Followed By Conversion to the imine

Acetonitrile (100 ml) was cooled to 0° C. after which DMF (0.88 g, 12.0 mmol, 0.93 mL) and oxalyl chloride (1.54 g, 12.0 mmol, 1.06 mL) were added. After 15 minutes' stirring at 0° C., subsequently a solution of the homoallylamine adduct from Example II (2.0 gram, 8.1 mmol) in acetonitrile (100 ml) was added. After 15 minutes' stirring pyridine was added (1.88 g, 24.0 mmol, 1.93 mL). After heating to room temperature and a further 15 minutes' stirring at room temperature water was added (200 ml) and the mixture was extracted-with dichloromethane (2×50 ml). Evaporation of the solvent produced an orange oil that consisted of a mixture of nitrile and imine.

This mixture was subsequently heated for five minutes with a heat gun (300° C.), resulting in a 44% imine yield (0.8 g, 3.5 mmol).

¹H NMR (200 MHz, CDCl₃): 8.17 (s, 1H), 7.73-7.78 (m, 2H), 7.25-7.42 (m, 3H), 5.62-5.79 (m, 1H), 5.00-5.09 (m, 2H), 2.86-2.95 (m, 1H), 2.39-2.48 (m, 2H), 1.87-1.97 (m, 1H), 0.92-0.98 (m, 6H).

¹³C NMR (50 MHz, CDCl₃): 158.0, 135.0, 134.9, 128.8, 127.5, 127.0, 126.7, 115.0, 75.8, 36.7, 31.2, 18.5, 17.2.

### Example IIIa

Dehydration of amide to nitrile with oxalylchloride/DMF and TEA

To CH₂Cl₂ (600 mL), cooled with an ice bath, was added DMF (144.0 mmol, 10.56 g, 11.16 mL). Oxalylchloride (144.0 mmol, 18.48 9, 12.72 mL) was added dropwise. After the formation of gas (CO and CO₂) had ceased, a solution of the amide of Example II (97.6 mmol, 24.0 g) in CH₂Cl₂ (100 mL) was added dropwise in 10 minutes. Triethylamine (95.5 mmol, 9.87 g, 13.48 mL) was added dropwise in 5 minutes and the reaction was stirred at room temperature for 30 minutes. H₂O (500 mL) was added and the organic phase was separated. The organic layer was dried over Na₂SO₄ and filtered. Evaporation of the solvent furnishes a red oil (22.0 gram). The red oil was dissolved in a mixture of ethyl acetate/Heptane (1/5) and was filtered through a short silica filter. Evaporation of the solvent at 30° C. under reduced pressure yields a yellow oil consisiting of pure product nitrile (17.4 gram, 78%). ¹H NMR (200 MHz, CDCl₃): δ7.3-7.6 (m, 5H), 5.7-5.9 (m, 1H), 5.0-5.2 (m, 2H), 4.7 (bs, 1H), 2.7-2.8 (m, 1H), 2.8-2.9 (m, 1H), 2.3-2.4 (m, 1H), 1.8-2.0 (m, 2H), 0.9-1.0 (m, 6H).

### Example IV

Hydrolysis of the amide of Example II to the carboxylic acid

The homoallylamine amide (3.0 g, 11.5 mmol) from Example II was added to 15% aqueous HCl (75 mL). The mixture was boiled for 3 hours. The water was evaporated. The product was isolated as HCl salt (light brown solid): 3.17 gram (10.7 mmol; 93%).

¹H NMR (200 MHz, DMSO-d6): 9.5 (s, 1H), 7.6 (m, 2H), 7.4 (m, 3H), 5.65 (m, 1H), 5.2 (m, 3H), 2.8 (m, 1H), 1.4 (m, 3H), 0.6 (m, 6H).

### Example V

Conversion of the carboxylic acid of Example IV to the imine

To CH₃CN (100 mL), cooled with an ice bath, was added DMF (15 mmol, 1.10 g, 1.16 mL). Oxalylchloride (15 mmol, 1.90 g, 1.30 mL) was added dropwise. After the formation of gas (CO and CO₂) had ceased, the carboxylic acid from Example IV (10 mmol, 2.49 gram) was added in one portion. The mixture was stirred for 15 minutes forming a clear yellow solution. TEA (30 mmol, 3.0 g, 4.2 mL) was added dropwise keeping the temperature below 10° C. A gas evolved (CO), the reaction mixture turned orange/yellow and a precipitate formed. The reaction mixture was stirred for 15 minutes. The ice bath was removed and H₂O (200 mL) was added. The mixture was extracted with Et₂O (2×50 mL). The Et₂O was evaporated and the residue was taken up in CHCl₃ (50 mL). The organic phase was washed once with H₂O (50 mL). The CHCl₃ was dried on MgSO₄ and filtered. Evaporation of the filtrate furnishes (R)-imine (yellow oil, 89%).

¹H NMR (200 MHz, CDCl₃): 8.16 (s, 1H), 7.66-7.69 (m, 2H), 7.34-7.36 (m, 3H), 5.61-5.75 (m, 1H), 4.92-4.99 (m, 2H), 3.09-3.27 (m, 1H), 2.27-2.31 (dd, 2H), 1.15-1.65 (m, 3H), 0.79-0.89 (m, 6H).

¹³C NMR (50 MHz, CDCl₃) d 156.9, 133.5, 127.9, 126.0, 125.6, 114.2, 66.7, 42.4, 38.9, 22.0, 21.2, 19.0.

### Example VI

Conversion of the imine of Examples III and V to the amine: with phenylhydrazine

The imine, 3.3 g (16.6 mmol), was dissolved in hexane (50 ml). 1.66 ml Phenylhydrazine (16.6 mmol) was added and the reaction mixture was stirred for 18 hours at room temperature. After filtration through a glass filter water was added and subsequently acidification with HCl (30%) took place. The hexane layer was separated and the aqueous phase made basic with NaOH (33%). The product was extracted with hexane (2×50 ml). The hexane solution was dried on MgSO4, filtered and evaporated. After evaporation the product was isolated as an orange oil: 1.7 g (15.3 mmol, 92%).

¹H NMR (200 MHz, CDCl₃): 5.61-5.83 (m, 1H), 5.02-5.12 (m, 2H), 2.53-2.60 (m, 1H), 2.20-2.26 (m, 1H), 1.87-2.01 (m, 1H), 1.58-1.62 (m, 1H), 0.86-0.92 (m, 6H).

¹³C NMR (50 MHz, CDCl₃): 134.6, 115.5, 54.2, 37.5, 37.50, 31.18, 17.50, 15.99.

### Example VII

Conversion of the imine of Examples III and V to the amine: with hydroxylamine

The imine (2.0 g, 9.9 mmol) was dissolved in 50% aqueous THF (50 ml). Hydroxylamine HCl salt (2.1 g, 29.8 mmol) was added and the reaction mixture was stirred for 18 hours at room temperature. The THF was evaporated under vacuum and the residue acidified with HCl (30%) to pH=1. The aqueous phase was subsequently extracted with ethyl acetate (2×50 ml). Then the aqueous phase was made basic with NaOH (33%) and the product was extracted with hexane (2×50 ml). The hexane solution was dried on MgSO₄, filtered and evaporated. After evaporation the product was isolated as a yellow oil: 0.68 g (5.9 mmol, 60%)

NMR data identical to Example VI.

### Example VIII

The crude mixture obtained in Example III was heated at 160° C. under vacuo for a few minutes until the nitrile was quantitatively converted into the (R)-imine (orange oil 44% rel. to amide). ¹H NMR (200 MHz, CDCl₃): δ8.11 (s, 1H), 7.68-7.71 (m, 2H), 7.27-7.37 (m, 3H), 5.61-5.70 (m, 1H), 4.91-4.99 (m, 2H), 2.81-2.87 (m, 1H), 2.33-2.41 (m, 2H), 1.82-1.89 (m, 1H), 0.86-0.91 (m, 6H). ¹³C NMR (50 MHz, CDCl₃) δ157.99, 135.01, 134.90, 128.74, 126.96, 126.65, 114.87, 75.79, 36.55, 31.14, 18.32, 17.11. MS (El) [m/e, %]: 201 [M⁺, 4.3]; 160 [M⁺-C₃H₅, 100].

### Example IX

The nitrile of Example IIIa (32.5 mmol; 7.4 g) was dissolved in ethanol (150 mL). K₂CO₃ (2 equivalents; 64.9 mmol; 8.97 gram) was added. The reaction mixture was refluxed for two hours. After cooling the reaction mixture to room temperature, the solvent was evaporated. The residue was mixed with water (100 mL) and CH₂Cl₂ (100 mL). The organic phase was separated, dried over Na₂SO₄ and filtered. The solvent was evaporated furnishing the imine as a yellow oil (6.2 g; 84%).

### Example X

The amide of Example II (25.2 mmol, 6.3 g) was mixed with H₂O (40 mL). At room temperature, solid Na₂O₂ (27.7 mmol, 2.2 g, 1.1 eq) was added and the mixture was refluxed for 18 hrs. The reaction mixture was cooled to room temperature and neutralized to pH=6-7 with aqueous HCl (30%). The precipitate was filtered off and dried (colorless powder, 84%, mixture of two diastereomers 60:40). Due to racemisation of the chiral auxiliary (phenylglycine) part, two diastereomers were formed. The amine obtained after removal of the auxiliary turned out to be enantiomerically pure. m.p. 75-76° C. ¹H NMR (200 MHz, DMSO): δ7.21-7.41 (m, 5H), 5.43-5.57 (m, 1H), 5.13-5.22 (m, 2H), 4.93-4.99 (m, 2H), 4.63 (s, 1H), 4.68 (s,1H), 1.84-2.5 (m, 4H), 0.72-0.90 (m, 6H).

### Example XI

Dehydration of amide to nitrile with triflic anhydride

To CH₂Cl₂ (250 mL), cooled with an ice bath, was added the amide of Example II (69.1 mmol, 14.0 g) and triethylamine (114.1 mmol; 11.8 g; 16.1 mL). Triflic anhydride (68.4 mmol, 19.3 g, 11.5 mL) was added dropwise in 5 minutes and the reaction was warmed to room temperature. The reaction mixture was stirred at room temperature for 30 minutes. H₂O (250 mL) was added and the organic phase was separated. The organic layer was dried over Na₂SO₄ and filtered. Evaporation of the solvent furnished an orange oil (11.9 g) consisting of a mixture of nitrile (80%) and imine (20%).

### Example XII

To a solution of the amide of Example II (18.8 mmol; 4.63 g) in CH₂Cl₂ (100 mL) cooled with an ice bath, was added triethylamine (37.6 mmol; 5.28 mL) and oxalylchloride (18.8 mmol; 1.64 mL). The color turned orange and a gas evolved. The reaction mixture was warmed to room temperature and water (100 mL) was added. The organic phase was separated. The organic layer was dried over Na₂SO₄ and filtered. Evaporation of the solvent furnishes a red oil (5.0 gram). The red oil was dissolved in a mixture of EtAc/Heptane (1/5) and was filtered though a short silica filter. Evaporation of the solvent at 30° C. under reduced pressure yields a yellow oil of the nitrile (1.5 gram, 32%).

### Example XIII

To a cooled (ice-bath) solution of N-benzylidene-DL-valine amide (29.5 mmol; 5 g, prepared from DL-valine amide and benzaldehyde) in THF (25 mL) was added a solution of allylzinc bromide (1.5 equivalent) in THF (25 mL), prepared from zinc wool (2.85 g) and allylbromide (3.8 mL). The reaction mixture was warmed to room temperature and was poured into 100 mL water. The product was extracted with ethylacetate (200 mL). The organic phase was dried over Na₂SO₄ and filtered. The solvent was evaporated leaving the racemic N-(1-phenyl-3-buten-1-yl)-valine amide as an oil in a diastereomeric ratio of >95:5. Upon addition of heptane, the oil solidified. Yield: 4.5 g, 76%. ¹H NMR (200 MHz, CDCl₃): δ7.2 (m, 5H), 6.4 and 5.8 (bs, 1H), 5.6 (m, 1H), 4.9 (m, 2H), 3.3 (t, 1H), 2.5 (m, 1H), 2.2 (m, 2H), 0.8-0.9 (m, 6H).

### Example XIV

To CH₂Cl₂ (50 mL), cooled with an ice bath, was added DMF (5.4, mmol, 0.39 g, 0.42 mL). Oxalylchloride (5.4 mmol, 0.69 g, 0.48 mL) was added dropwise. After the formation of gas (CO and CO₂) had ceased, was added the N-(1-phenyl-3-buten-1-yl)-valine amide from example XIII (3.6 mmol, 0.89 g) all at once. Triethylamine (3.6 mmol, 0.51 mL) was added dropwise in 5 minutes and the reaction was stirred at room temperature for 30 minutes. H₂O (50 mL) was added and the organic phase was separated. The organic layer was dried over Na₂SO₄ and filtered. Evaporation of the solvent furnished the corresponding amino nitrile product as a yellow oil (0.78 g; 95%). ¹H NMR (200 MHz, CDCl₃): δ7.3 (m, 5H), 5.6-5.9 (m, 1H), 5.1-5.2 (m, 2H), 3.9 (dd, 1H), 3.0 (d, 1H), 2.2-2.6 (m, 2H), 1.9 (m, 1H), 1.6 (bs, 1H), 1.0 (m, 6H).

### Example XV

The amino nitrile from example XIV (3.4 mmol; 0.78 g) was dissolved in ethanol (50 mL). K₂CO₃ (2 equivalents; 6.8 mmol; 0.93 gram) was added. The reaction mixture was refluxed overnight. After cooling the reaction mixture to room temperature, the solvent was evaporated. The residue was mixed with water (50 mL) and DCM (50 mL). The organic phase was separated, dried over Na₂SO₄ and filtered. The solvent was evaporated furnishing 0.6 gr of a yellowish oil. According to ¹H-NMR spectroscopy, the crude product contained a mixture of N-i-butylidene-1-amino-1-phenylbutene-3 (82%) and the starting amino nitrile (18%). ¹H NMR of the imine (200 MHz, CDCl₃): δ7.6 (d, 1H), 7.3-7.4 (m, 5H), 5.7 (m, 1H), 5.0 (m, 2H), 4.0 (t, 1H), 2.4-2.6 (m, 3H), 1.0 (m, 6H).

### Example XVI

The imine obtained in example XV (0.5 g, 2.5 mmol) was dissolved in 50% aqueous THF (50 mL). To this solution 3 equivalents of NH₂OH.HCl (0.52 g, 7.45 mmol) were added and the reaction mixture was stirred overnight at ambient temperature. The THF was evaporated under reduced pressure and the residue was treated with aqueous HCl (30%) until pH=1. The aqueous phase was extracted with EtOAc. The water phase was adjusted to pH=10 with aqueous NaOH (33%) and extracted with CH₂Cl₂. After drying over Na₂SO₄, the solvent was evaporated furnishing 1-amino-1-phenylbutene-3. (colourless oil, 76%). ¹H NMR (200 MHz, CDCl₃): δ7.25 (m, 5H), 5.8 (m, 1H), 5.1 (m, 2H), 4.0 (m, 1H), 2.4 (m, 2H).

1. Process for removing a residual fragment of a chiral auxiliary in the preparation of an enantiomerically enriched, amine-functionalized compound with formula 1 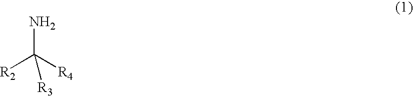
in which R₂, R₃, R₄ are each different and stand for H, a substituted or unsubstituted (cyclo)alkyl group, alkenyl group, aryl group, cyclic or non-cyclic heteroalkyl or heteroaryl group with one or more N—, O—or S-atoms, or (CH₂)n-COR₆, where n=1, 2, 3 . . . 6 and R₆═OH, a substituted or unsubstituted alkyl group, aryl group, alkoxy group or amino group, in which a diastereomeric compound with formula 2 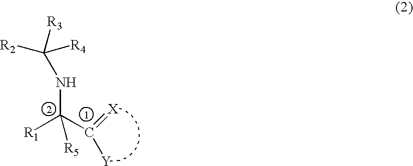
in which R₂, R₃ and R₄ are as defined above, and R₁ and R₅ are each different and R₁ stands for a modified or unmodified side tail of a proteogenous amino acid or a substituted or unsubstituted phenyl group, R₅ stands for H or a lower alkyl group, and in which X═O and Y═OR, where R represents H or a C₁-C₇ alkyl group, or NR₇R₈, where R₇ and R₈ each independently represent H, a (cyclo)alkyl group, alkenyl group or aryl group, or X and Y together stand for N, is subjected to a non-reductive removal of the residual fragment of the chiral auxiliary, the carbon atom which is removed having an oxidation state of +3 which is not lowered during the method of removal.

2. Process according to claim 1, in which a compound with formula (3) is formed upon the non-reductive removal of the residual fragment 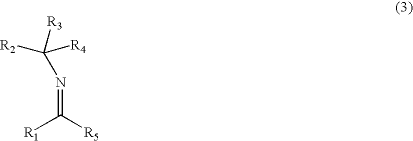
in which R₁, R₂, R₃, R₄ and R₅ are as described above and subsequently the compound with formula (3) is converted (in a known way) into the corresponding amine-functionalized compound.

3. Process according to claim 1 or 2, in which the chiral auxiliary is chosen from the group of amides or esters of proteogenous amino acids.

4. Process according to any one of claims 1-3, in which the chiral auxiliary is chosen from the group of phenylglycine amide, an ester of phenylglycine, p-OH-phenylglycine amide, an ester of p-OH-phenylglycine, αmethylphenylglycine amide and an ester of α-methylphenylglycine.

5. Process according to any one of claims 1-4, in which the residual fragment originates from an amino acid amide of which the amide group is not substituted and the residual fragment is removed via dehydration of the amide group to a nitrile group, followed by a retro-Strecker reaction in which an imine is formed and conversion of the imine into the corresponding chiral amine-functionalized compound.

6. Process according to claim 5 wherein the dehydration of the amide group to the nitrile group is performed by treating the amide with a Vilsmeier reagent.

7. Process according to any one of claims 1-4, in which the residual fragment originates from an amino acid amide and the residual fragment is removed via hydrolysis of the amide group to a carboxyl group, followed by a reaction that, overall, leads to removal of the CO₂ group, in which an imine is formed, and conversion of the imine into the corresponding chiral amine-functionalized compound.

8. Process according to any one of claims 1-4, in which the residual fragment originates from an ester of an amino acid and the residual fragment is removed via conversion of the ester with the aid of ammonia to the corresponding amide after which the residual fragment is removed according to claim 5 or 6.

9. Process according to any one of claims 1-4, in which the residual fragment originates from an ester and the residual fragment is removed via hydrolysis of the ester group to a carboxyl group followed by a reaction that, overall, leads to removal of the CO₂ group, in which an imine is formed, and conversion of the imine into the corresponding chiral amine-functionalized compound.

10. Process according to any one of claims 1-4, in which the residual fragment originates from an amino acid amide of which the amide group is not substituted, the residual fragment being removed via dehydration of the amide group to a nitrile group, followed by a treatment with an alcohol and an acid, upon which an ester group is formed after which the residual fragment is removed according to claim 7 or 8.

11. Process according to any one of claims 1-10, in which first a compound with formula 2, in which R₁, R₂, R₃, R₄, R₅, X and Y are as described above, is prepared by converting an enantiomerically enriched amino acid derivative with formula 4 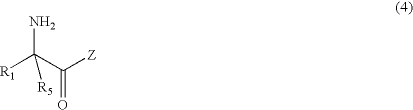
in which R₁ and R₅ have the above-mentioned meanings and in which Z stands for OH, a C₁-C₇ alkoxy group or NR₇R₈, with R₇ and R₈ each independently representing H, a (cyclo)alkyl group, alkenyl group or aryl group, with the aid of a compound with formula 5
R₂—C(O)—R₃ (5)
where R₂ and R₃ have the above-mentioned meanings, into the corresponding Schiff base and subsequently converting the resulting Schiff base into the enantiomerically enriched compound with formula 2 with the aid of a reducing agent or an organometallic compound.

12. Process according to any one of claims 1-11, in which the enantiomerically enriched amine-functionalized compound obtained is subsequently used in the preparation of agrochemicals or pharmaceuticals.
